(19) United States
(12) Patent Application Publication
(10) Pub. No.: US 2024/0228944 A9
(43) Pub. Date: Jul. 11, 2024

(54) METHOD FOR PREPARING BACTERIAL PRODUCTS

(71) Applicant: Chr. Hansen A/S, Hoersholm (DK)

(72) Inventors: Zuzana Mladenovska, Hoersholm (DK); Surender Kumar Dhayal, Hoersholm (DK); Kim Nielsen, Hoersholm (DK)

(73) Assignee: Chr. Hansen A/S, Hoersholm (DK)

(21) Appl. No.: 18/254,658

(22) PCT Filed: Nov. 29, 2021
(86) PCT No.: PCT/EP2021/083330

§ 371 Date: May 26, 2023

(30) Foreign Application Priority Data
Nov. 30, 2020 (EP) 20210789.2

(51) Int. Cl. C12N 1/04; C12N 1/20

(52) U.S. Cl. CPC C12N 1/04; C12N 1/20

(57) ABSTRACT

The present invention relates to the field of frozen or dry compositions for prokaryotes, in particular fermentative bacteria such as lactic acid bacteria, a method for preparing frozen or dry prokaryotic compositions and compositions which may be prepared by said method. The cells in a cell concentrate are activated by allowing them to ferment a carbohydrate, before freezing and/or drying to provide a product. The hydrophobicity of the cell surface increases during activation, and this has been found to increase the storage stability of the product.

The present invention relates to the field of frozen or dry compositions for certain bacteria, in particular fermentative bacteria such as lactic acid bacteria, a method for preparing frozen or dry bacterial compositions and compositions which may be prepared by said method.

### BACKGROUND OF THE INVENTION

Fermentative bacteria are anaerobic bacteria in the metabolism of which an organic compound (instead of oxygen) is the terminal electron (or hydrogen) acceptor. Based on the pattern of products formed in fermentations, bacteria are classified as homofermentative and heterofermentative. Lactic acid bacteria (LAB) with homofermentative metabolism, produce lactic acid as the major or sole product of sugar fermentation. Examples of homofermentative lactic acid bacteria are species Lactococcus lactis, Lactobacillus delbrueckii subsp. bulgaricus or Streptococcus thermophilus. Heterofermentative bacteria produce various products from fermentation of sugars and the end products depends on the type of sugar served in fermentation. Heterofermentative lactic acid bacteria, such Oenococcus, Leuconostoc and some Lactobacillus species, such as Lactobacillus reuteri, ferment sugars in addition to lactate, CO₂ and ethanol, also to acetate and polyols. The present invention is applicable to both types of fermentative bacteria.

Fermentative bacteria are involved in numerous industrially relevant processes. For instance, bacterial cultures, in particular cultures of bacteria that are generally classified as LAB, are essential in the making of all fermented milk products, cheese and butter. Cultures of such bacteria may be referred to as starter cultures and they impart specific features to various dairy products by performing a number of functions.

Many lactic acid bacteria are known to have probiotic properties (i.e. they have a beneficial health effect on humans and animals when ingested). Probiotics are widely applied in dry form. In most cases, it is imperative that the microorganisms remain viable after prolonged storage of dried products, in order for these to impart their beneficial effect. For instance, if the LAB composition is mixed with milk powder to make a suitable infant powder, one generally needs a very storage stable LAB composition, essentially because an infant powder product may be given to infants quite a long time after its actual fabrication date. Accordingly, if the infant powder is given to infants e.g. 30 weeks (or later) after its actual fabrication date, it is evident that the LAB composition incorporated into the infant powder should be quite storage stable in order to maintain viability of the LAB cells.

Since it is well known that bacteria can easily lose viability upon exposure to various stresses, it is a general practice in industrial production of bacterial cultures to use additives. These additives are supposed to protect cells during different steps of a production process and later on during shelf storage of dried bacteria. Bacteria that are to be frozen or dried, for example spray-dried, freeze-dried, vacuum-dried, are mixed as a cell suspension with additives and then processed in a sequence of various technological steps. The role of the additive is to protect the bacterial cell composition during freezing (so called cryo-protectants), drying or freeze-drying (so called lyo-protectants). However, certain damage of cells during these processes cannot be avoided (Coulibaly et al. (2018) ARRB 24 (4): 1-15)

Additives can be composed of a single compound (Hubalek (2003) Cryobiology 46, 205-229) or of mixtures of protective agents. For example, WO 2010/138522 (Advanced Bionutrition Corporation) describes a LAB cell culture composition that is said to be useful to be incorporated into an infant powder product. A preferred composition comprises alginate, inulin, trehalose and hydrolyzed protein (see table 1, paragraph [0094]). WO 2013/001089 (Chr. Hansen) discloses a dry LAB composition comprising trehalose, inulin and casein. Carvalho et al (2004) Biotechnol Prog. 20, 248-254, discloses the effects of various sugars added to growth and drying media upon thermotolerance and survival throughout storage of freeze-dried Lactobacillus delbrueckii ssp. bulgaricus.

Bacterial products can also be formulated as frozen products. For example, commercial starter cultures may be distributed as frozen cultures. Highly concentrated frozen cultures, particularly when prepared as pellets, are commercially very useful since such cultures can be inoculated directly into the fermentation medium (e.g. milk or meat) without intermediate transfer. In other words, such highly concentrated frozen cultures comprise bacteria in an amount that makes in-house bulk starter cultures at the end-users superfluous. A “bulk starter” is defined herein as a starter culture propagated at the food processing plant for inoculation into the fermentation medium. Highly concentrated cultures may be referred to as direct vat set (DVS)-cultures. In order to comprise sufficient bacteria to be used as a DVS-culture at the end-users, a concentrated frozen culture generally has to have a weight of at least 50 g and a content of viable bacteria of at least 10⁹ colony forming units (CFU) per g. WO 2005/080548 (Chr. Hansen) discloses pellet-frozen lactic acid bacteria (LAB) cultures that are stabilised with, for example, a mixture of trehalose and sucrose and do not form clumps when stored.

The prior art discloses maintaining the cell culture at 4° C. during all intermediate steps of the process, including during the step of formulating the cell concentrate with additive, with the aims of limiting the cell degradation reactions. (Fonseca et al. (2015) Chapter 24: Freeze-Drying of Lactic Acid Bacteria in Wolkers & Oldenhof (Eds), Cryopreservation and Freeze-drying Protocols, Third edition).

In prior art processes, a concentrated bacterial culture is obtained by known methods of culturing the bacteria in a growth medium and then concentrating the culture, for example by centrifugation, with the bacteria being separated from the growth medium. The concentrated culture is then admixed with the desired preservative(s) and, shortly thereafter, the resulting mixture is frozen or dried.

The microbial cell surface has a very complex composition and it plays a key role in interactions between microorganisms and the surrounding environment (Burgain J, et al (2014) Advances in Colloid and Interface Science 213, 21-35).

The cell wall of Gram-positive bacteria consists of a peptidoglycan layer with embedded teichoic, lipoteichoic acid and cell wall polysaccharides. The peptidoglycan layer can be covered by a proteinaceous S-layer and decorated by various polysaccharides (Zeidan et al 2017, FEMS Microbiology Reviews 41: 168-200). The surface of Gram-negative bacteria is different. It is made of capsular polysaccharides which are decorated with various polymeric substances such as carbohydrates, lipo-oligosaccharides and lipopolysaccharides. This complex composition of cell surface can be captured by physicochemical analyses such as measurement of cell surface interactions by hydrophobicity analysis and cell surface charge determined by zeta potential. Particularly, the combination of these two assays with advanced microscopy techniques has contributed to a more profound characterization of the cell wall of lactic acid bacteria (Schär-Zammaretti and Ubbink (2003) Biophysical Journal 85, 4076-4092). The hydrophobicity analysis, originally developed by Rosenberg et al. (1980, FEMS Microbiology Letters 9, 29-33) as a measurement of bacterial cell adherence to liquid hydrocarbon, was refined by Schär-Zammaretti and Ubbink (op. cit.) into determination of interfacial adhesion curves, reflecting partitioning of bacteria from aqueous phase to hexadecane in organic phase. From the pattern of the interfacial adhesion curves and zeta potential it is possible to differentiate between the primary constituents of the cell surface. The presence of surface proteins was found to be correlated with elevated isoelectric point and high hydrophobicity of surface. Teichoic acid made the surface hydrophobic and strongly negatively charged. A high abundance of polysaccharides rendered the cell surface hydrophilic and weakly charged.

The characterization of cell surface properties and links to the growth conditions of bacteria have been the subjects of numerous studies (Schär-Zammaretti et al (2005) AEM 71, 8165-8173; and Millsap K-W et al (1996) J. Microbiol. Methods 27, 239-242). These studies demonstrated that the composition of the growth medium in a fermentation process had a significant impact not only on the cell yield, but also on the cell surface properties of lactic acid bacteria.

In industrial processes for the production of beneficial bacteria, it is important that bacterial cells exhibit a high degree of robustness and maintain viability after fermentation, during several steps in the downstream processes. The link between the physicochemical characteristics of cells and cell survival in the downstream process has been described solely in the study of Zupancic et al. (Pharmaceutics 2019, 11, 483; doi:10.3390/pharmaceutics11090483). In this work it was demonstrated that lactic acid bacteria with hydrophobic cell surface survived better the process of electrospinning than bacteria with hydrophilic cell surface. To the best of our knowledge, the correlation between the composition of the microbial cell surface and the storage stability of frozen, dried or freeze-dried products has not yet been discovered. It has not yet been published that the cell surface properties can be modulated in a post-fermentation process in highly concentrated suspensions of non-growing microbial cells by conditioning of cells with nutrients. It has not been proven that a link exists between activation of cells and modification of cell surface in the downstream process and stability of frozen, dried or freeze-dried product.

### SUMMARY OF THE INVENTION

The invention is derived from the discovery that it is beneficial to stimulate the bacteria in the concentrated culture such that they start to metabolise a fermentable carbohydrate. A non-fermentable protectant may also be included in the concentrated culture or added later, as is known. The concentrated culture is typically held for between 30 minutes and 8 hours to allow the cells to metabolise at least part of the fermentable carbohydrate. This has the short term effect of activating the cells in order to modify the cell surface, and the longer term effect of stabilising them under storage conditions when frozen and/or dried. The invention will now be defined in more detail.

The invention provides a method of preparing a frozen, dried or freeze-dried product comprising an asporogenous prokaryote, the method comprising the steps of:
(i) combining a cell concentrate of the prokaryote with a medium comprising at least one carbohydrate, the carbohydrate being fermentable by the prokaryote, to form a pre-processing composition;
(ii) holding the pre-processing composition for a period of time and at a temperature sufficient to allow the prokaryote to ferment the fermentable carbohydrate and form an activated composition;
(iii) optionally adding a protective compound to the activated composition; and
(iv) preserving the activated composition by
   (a) freezing the activated composition to form a frozen prokaryote product;
   (b) drying the activated product to form a dried prokaryote product; or
   (c) freezing the activated composition to form a frozen prokaryote intermediate product and then lyophilising the intermediate product to form a freeze-dried prokaryote product.

A fermentation broth will usually have 5E+08 to 1E+11 total cells/g fermentation broth, where ‘total cells’ means viable and non-viable cells and the weight of the fermentation broth includes the cells suspended in it. The concentration of cells in a liquid can be measured by standard techniques such as the Petroff Hausser counting chamber method or flow cytometry.

A concentrated culture (“cell concentrate”) is generally formed by separating the cells from a fermentation broth with a concentration factor of 2× to 90×, typically 5× to 60×, for example 10× to 50× or 20× to 40×. The total concentration of cells in the cell concentrate will therefore be in the range 1E+09 to 9E+12 prokaryote cells/g, preferably 2.5E+09 to 3E+12 prokaryote cells/g, 1.3E+10 to 2E+12 prokaryote cells/g, 2E+10 to 1.3E+12 prokaryote cells/g, 3E+10 to 2.5E+11 prokaryote cells/g, or 4.5E+10 to 1E+11 prokaryote cells/g.

The proportion of dry matter in a cell concentrate is typically 8-25%, for example 10-20%, such as about 13%, 14%, 15% or 16%.

The carbohydrate may, for example, be one or more of: a monosaccharide such as glucose, fructose, galactose or mannose; a disaccharide such as sucrose, trehalose, maltose or lactose; a sugar alcohol such as inositol; a trisaccharide such as maltotriose or raffinose; an oligosaccharide such as a fructooligosaccharide or such as a maltodextrin with DE 3-20; and a polysaccharide such as starch or inulin.

The total concentration of the carbohydrate in the pre-processing composition is preferably 1-90% w/w, preferably 1-50% w/w, 1-20% w/w, 1-15% w/w, or 1-10% w/w.

The protective compound may be a cryoprotectant and/or a lyoprotectant and/or a storage stabiliser, such as gum arabic, a maltodextrin, starch, pectin, cellulose, xylan, or a polyol such as glycerol, sucrose, trehalose or maltose, a protein such as gelatin, a peptide such as are supplied by yeast extract, an amino acid such as proline or a sugar alcohol such as sorbitol, mannitol or inositol.

The concentration of the protective compound in the pre-processing composition is preferably 5-90% w/w, preferably 5-50% w/w, 5-30% w/w, 5-15% w/w, or 5-10% w/w.

The carbohydrate is, for example, glucose and the protective compound is, for example, gum arabic, and the combined concentration of the carbohydrate and the protective compound in the pre-processing composition is preferably 5-20% w/w, for example 10-20% w/w, more preferably 12-15% w/w and most preferably about 13-14% w/w.

Likewise, the carbohydrate can be glucose and the protective compound can be gum arabic, and the concentration of the carbohydrate in the pre-processing composition is 1-12%, preferably 1-10%, and the concentration of the gum arabic in the pre-processing composition is 10-35% w/w, preferably 10-22% w/w, more preferably 10-15% w/w.

Preferably, step (ii) lasts for 0.5 to 8 hours, more preferably 2-4 hours, and is best carried out at 4° C. to 20° C., preferably 10-15° C.

In step (ii), the cells should become activated. This can be determined by reference to the hydrophobicity of the cell surface. At the end of step (ii), in activated cells, the hydrophobicity of the cell surface is at least 20%, preferably at least 50%, and/or during step (ii) it has increased (compared with the hydrophobicity at the start of step (ii)) by at least 20%, preferably at least 30%, 40%, 50%, 60%, 70%, 80%, 90% or 100%, as measured by the MATH method at 22° C. and expressed as [(Initial OD₆₀₀−Final OD₆₀₀)/Initial OD₆₀₀]*100 when measured with a Φ[V_{H}/V_{B}] at a point between 0.01 and 1.0 and the initial OD₆₀₀ (nm) is 0.5. That is to say, at at least one point in the range 0.01 to 1.0, the hydrophobicity is as stated. Preferably, the hydrophobicity is as stated at at least three of Φ[V_{H}/V_{B}]=0.01, Φ[V_{H}/V_{B}]=0.05, Φ[V_{H}/V_{B}]=0.1, Φ[V_{H}/V_{B}]=0.5 and Φ[V_{H}/V_{B}]=1.0, and particularly at Φ[V_{H}/V_{B}]=0.5 and/or Φ[V_{H}/V_{B}]=1.0.

In terms of an increase in hydrophobicity, the starting value and the finishing value should be measured at the same Φ[V_{H}/V_{B}] value.

The metabolism of most fermentable carbohydrates will result in the formation of an acid. For example, in the case of lactic acid bacteria, the acid is lactic acid. During activation of the cells in step (ii), the pH ideally decreases to no more than 4.5, preferably no more than 4.0, and/or decreases by at least 0.1 pH units, preferably at least 0.2, 0.5, 1.0, 1.5 pH or 2.0 units.

Alternatively, or as well, during activation of the cells in step (ii), the isoelectric point (pI) of the cells increases to at least 3.0, preferably at least 3.3, and/or increases by at least 0.1, preferably at least 0.2, 0.5, 1.0, 1.5 pH or 2.0 units, but in either case is preferably less than 3.8, still more preferably less than 3.6 or less than 3.5.

Activation of the cells is indicated by at least one of (a) the increase in hydrophobicity, (b) the reduction of pH and (c) the increase in pI, preferably two of those phenomena, and ideally all three. Cells that are already sufficiently hydrophobic may not need activation and so the process of the invention need not be employed.

At the end of step (ii), a stabilising amount of the fermentable carbohydrate may still present in the activated composition. That is to say, the metabolism of the carbohydrate has achieved the desired activation of the cells but there is enough left over for the cells to be additionally stabilised during the processing and/or storage of the product. Alternatively, the fermentable carbohydrate is fully converted in step (ii) and nothing is left from it in the formulated cell concentrate at the end of holding time, but it has been metabolised into products that will contribute to the stabilisation during processing and/or storage. This can be the case for formulations of heterofermentative bacteria with an additive containing fermentable sucrose. In this particular case, sucrose will be converted to mannitol, lactate, acetate, carbon dioxide and ethanol.

The compound with a protective function in the downstream process is mannitol. The dried product will be stable, due to the presence of mannitol, while sucrose is absent. Alternatively, if not enough of the fermentable carbohydrate or stabilising metabolites thereof remains, then a protective compound can be added at that stage. The protective compound can be fermentable (in which case either step (ii) then ends and the cells are frozen and or dried, or, if step (ii) continues, then enough protective compound is added such that enough remains for it to provide the desired protective function) or non-fermentable, in which case the activation step can continue, since the level of the protective compound will be unaffected. Thus, the non-fermentable protective compound is added at the start of, during or at the end of step (ii).

Preferably, the frozen prokaryote product or the frozen prokaryote intermediate product has a dry weight ratio of the final additive (i.e. the sum of the medium comprising at least one fermentable carbohydrate from step (i) and the protective compound from step (iii)) to cell concentrate of between 6:1 and 0.3:1, preferably between 3:1 and 0.5:1 and most preferably between 2:1 and 1:1.

The method of the invention is widely applicable. The prokaryote may be a fermentative bacterium
from the phylum Firmicutes, such as:
a lactic acid bacterium (LAB), preferably of a genus selected from the group consisting of Streptococcus (such as Streptococcus thermophilus), Lactococcus (such as Lactococcus lactis), Oenococcus (such as Oenococcus oeni), Leuconostoc (such as species Leuconostoc mesenteroides, Leuconostoc pseudomesenteroides), Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus; Eubacterium (such as Eubacterium limosum, Eubacterium aggregans, Eubacterium barkeri, Eubacterium lentum), Roseburia (Roseburia intestinalis, Roseburia hominis, Roseburia inulinivorans, Roseburia faecis and Roseburia cecicola), Faecalibacterium (such as species Faecalibacterium prausnitzii), Anaerostipes (such as Anaerostipes caccae), Anaerobutyricum (such as species Anaerobutyricum hallii, Anaerobutyricum soehngenii)
from the phylum Actinobacteria, such as genus Bifidobacterium (such as species Bifidobacterium animalis, Bifidobacterium longum, Bifidobacterium adolescentis, Bifidobacterium breve), genus Propionibacterium (such as species Propionibacterium freudenreichii), Cutibacterium (such as Cutibacteriun acnes)
from the phylum Bacteroidetes, such as genera Bacteroides (such as species Bacteroides fragilis, Bacteroides xylanisolvens), genus Prevotella (such as species Prevotella copri) or Alistipes
or from the phylum Verrucomicrobia, such as an Akkermansia (such as species Akkermansia muciniphila).

In particular, the prokaryote can be one or more of: Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

The invention furthermore provides a frozen or dried product comprising a non-sporulating prokaryote, obtainable by the method described above.

The potency of the frozen product can be 1E+09-1E+12 CFU/g; and the potency of the freeze-dried product can be 1E+09-1E+13 CFU/g.

The method is applicable to vegetative cells of non-spore-forming prokaryotic microorganisms from the domain Bacteria and Archaea. The invention relates to a broad spectrum of non-sporulating microorganisms used in food- and feed-producing industries, agriculture, medicine, for production of biofuels and biobased chemicals.

Non-spore-forming bacteria can be identified within the phyla Firmicutes, Actinobacteria and Bacteroidetes. The invention is particularly applicable to homo- and heterofermentative lactic acid bacteria in the Firmicutes phylum, and to bifidobacteria and propionibacteria in the Actinobacteria phylum. The invention is also applicable to obligate anaerobes of the class Clostridia in the Firmicutes phylum, such as fermentative, butyrate-producing bacteria of the genera Roseburia (e.g. Roseburia hominis and Roseburia inulinivorans), Anaerobutyricum hallii, Anaerobutyricum soehngenii), Eubacterium (e.g. Eubacterium limosum), Anaerostipes (e.g. Anaerostipes caccae), and Faecalibacterium (e.g. F. prausnitzii) which represent the core microbiota of human intestinal tract and are candidates for next generation of probiotics.

The industrially most useful lactic acid bacteria are found among Lactococcus species, Streptococcus species, Enterococcus species, Lactobacillus species (including all those that were classed as Lactobacillus until 2020), Leuconostoc species, Oenococcus, Bifidobacterium species, Propionibacterium and Pediococcus species. Accordingly, in a preferred embodiment the lactic acid bacteria are selected from the group consisting of these lactic acid bacteria.

The lactic acid bacteria are preferably of a genus selected from the group consisting of Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus. In particular, they can be Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, and/or Lactiplantibacillus pentosus. Others include Lactococcus lactis subsp. lactis, Lactococcus lactis subsp. cremoris, Leuconostoc lactis, Leuconostoc mesenteroides subsp. cremoris, Pediococcus pentosaceus, Lactococcus lactis subsp. lactis biovar. diacetylactis, Streptococcus thermophilus, Enterococcus, such as Enterococcus faecium, Bifidobacterium animalis subsp. lactis, Bifidobacterium animalis subsp. animalis, Bifidobacterium longum, Bifidobacterium adolescentis, Bifidobacterium breve, Lactobacillus helveticus, Lactobacillus fermentum, Lactobacillus salivarius, Lactobacillus delbrueckii subsp. bulgaricus and Lactobacillus acidophilus.

The composition may comprise one or more strains of lactic acid bacteria which may be selected from the group comprising: BB-12 ® (Bifidobacterium animalis subsp lactis BB-12 ®), DSM 15954; ATCC 29682, ATCC 27536, DSM 13692, DSM 10140, LA-5 (Lactobacillus acidophilus LA-5®, DSM 13241, LGG® (Lactobacillus rhamnosus LGG®, ATCC 53103, GR-1® (Lactobacillus rhamnosus GR-1®, ATCC 55826, RC-14® (Lactobacillus reuteri RC-14®), ATCC 55845, L. casei 431 ® (Lactobacillus paracasei subsp. paracasei L. casei 431®), ATCC 55544, F19® (Lactobacillus paracasei F19 ®, LMG-17806, TH-4® (Streptococcus thermophilus TH-4®), DSM 15957, PCC® (Lactobacillus fermentum PCC®), NM02/31074, and LP-33 ® (Lactobacillus paracasei subsp. paracasei LP-33 ®), CCTCC M204012.

The LAB culture may be a “mixed lactic acid bacteria (LAB) culture” or a “pure lactic acid bacteria (LAB) culture”. The term “mixed lactic acid bacteria (LAB) culture”, or “LAB” culture, denotes a mixed culture that comprises two or more different LAB species. The term a “pure lactic acid bacteria (LAB) culture” denotes a pure culture that comprises only a single LAB species. Accordingly, in a preferred embodiment the LAB culture is a LAB culture selected from the group consisting of these cultures.

The LAB culture may be washed, or non-washed, before mixing with the protective agents.

Preferably, the LAB cell is a probiotic cell.

The frozen or dried cells can be mixed with any suitable excipients to make blends, for example human food and animal feed compositions. For example, the cells can be mixed with milk powder to make an infant milk formula powder.

### DESCRIPTION OF THE FIGURES

FIG. 1A shows the interfacial adhesion curve of a crude cell concentrate of Lactobacillus animalis CHCC10506. All data points are the averages of three measurements with standard deviations.

FIG. 1B shows the Zeta potential of an Lactobacillus animalis CHCC10506 cell concentrate as a function of pH. All data points are the averages of three measurements with standard deviations.

FIG. 2 shows the CFU log loss in freeze-dried granulates of Lactobacillus animalis CHCC10506 during storage at 37° C. Various compositions in the legend refer to the concentration of single saccharides or polyol in formulated cell concentrate prior to freeze-drying.

FIG. 3 shows the CFU log loss in freeze-dried granulates of Lactobacillus animalis CHCC10506 during storage at 37° C. Various compositions in the legend refer to the concentration of carbohydrate mixture in formulated cell concentrate prior to freeze-drying. Gum arabic alone is included for direct comparison.

FIG. 4A shows the CFU log loss in freeze-dried granulates of Lactobacillus animalis CHCC10506 during storage at 37° C. Formulated cell concentrate prior to freeze-drying contained a mixture of 6.7% gum arabic and 6.7% of various fermentable carbohydrates, namely glucose, lactose, FOS, Glucidex® IT 12 or inositol.

FIG. 4B shows interfacial adhesion curves of freeze-dried Lactobacillus animalis CHCC10506 made from formulations of cell concentrate in a mixture of 6.7% gum arabic and 6.7% various fermentable carbohydrates or inositol prior to freeze-drying.

FIG. 4C shows Zeta potential as a function of pH for freeze-dried Lactobacillus animalis CHCC10506 made from formulations of cell concentrate in a mixture of 6.7% gum arabic and 6.7% various fermentable carbohydrates or inositol prior to freeze-drying.

FIG. 5A shows the CFU log loss during storage of freeze-dried Lactobacillus animalis CHCC10506 at 37° C. Cell concentrate was formulated in a mixture of 6.7% gum arabic and 6.7% glucose and held at differing temperatures during the holding period.

FIG. 5B shows interfacial adhesion curves of freeze-dried Lactobacillus animalis CHCC10506, made from cell concentrate formulations in a mixture of 6.7% gum arabic and 6.7% glucose, held at differing temperatures during the holding period.

FIG. 5C shows Zeta potential as a function of pH for freeze-dried Lactobacillus animalis CHCC10506, made from cell concentrate formulations in a mixture of 6.7% gum arabic and 6.7% glucose, held at differing temperatures during the holding period.

FIG. 6 shows the CFU log loss during storage at 37° C. of freeze-dried Lactobacillus animalis CHCC10506, made from cell concentrate formulations in a mixture of 6.7% gum arabic and 6.7% glucose, held for differing periods at 10° C.

FIG. 7 shows the CFU log loss during storage at 37° C. of freeze-dried Lactobacillus animalis CHCC10506, made from cell concentrate formulations in a mixture of 6.7% gum arabic and 6.7% Glucidex IT12, held for differing periods at 10° C.

### Definitions

“Fermentable”—a fermentable carbohydrate is one that can be metabolised by the bacterium. When such metabolism produces an acid, the pH in the culture during step (ii) decreases by at least 0.1 pH units, preferably at least 0.2, 0.5, 1.0, 1.5 pH or 2.0 units.

Fructo-oligosaccharides (FOS), also known as oligofructose or oligofructan, are mixtures of oligosaccharide fructans. FOS can be produced by degradation of inulin, or polyfructose, a polymer of D-fructose residues linked by β(2→4) bonds with a terminal α(1→2) linked D-glucose. The degree of polymerization of inulin ranges from 10 to 60. Inulin can be degraded enzymatically or chemically to a mixture of oligosaccharides with the general structure Glu-Fruₙ (abbrev. GFₙ) and Fruₘ (Fₘ), with n and m ranging from 1 to 7. This process also occurs to some extent in nature, and these oligosaccharides can be found in a large number of plants, especially in Jerusalem artichoke, chicory and the blue agave plant. The main components of commercial products are kestose (GF₂), nystose (GF₃), fructosylnystose (GF₄), bifurcose (GF₃), inulobiose (F₂), inulotriose (F₃), and inulotetraose (F₄). The second class of FOS is prepared by the transfructosylation action of a β-fructosidase of Aspergillus niger or Aspergillus on sucrose. The resulting mixture has the general formula of GFₙ, with n ranging from 1 to 5. Contrary to the inulin-derived FOS, as well as β(1→2) binding, other linkages do occur, however in limited numbers. In this patent application, “FOS” and cognate terms are used to describe the second class of FOS.

### EXAMPLES

The examples involve Lactobacillus animalis CHCC10506, deposited under accession number DSM 33570 at German Collection of Microorganisms and Cell Cultures GmbH (Braunschweig, Germany).

Lactobacillus animalis CHCC10506 was grown by fermentation in MRS medium (BD Difco™ Lactobacilli MRS Broth, Fisher Scientific). It contains Proteose Peptone No. 3 10 g/L, beef extract 10 g/L, yeast extract 5 g/L, dextrose 20 g/L, polysorbate 80 1 g/L, ammonium citrate 2 g/L, sodium acetate 5 g/L, magnesium sulfate 0.1 g/L, manganese sulfate 0.05 g/L, dipotassium phosphate 2 g/L in Milli-Q water. The inoculum for fermentation was prepared by growing the strain in a closed bottle with MRS medium, under static conditions, without pH control at 37° C. The incubation period was 7 hours. Fermentation was initiated by inoculation of 1% of pre-culture to the fermenter. Fermentation was carried out in MRS media under anaerobic conditions with nitrogen in the headspace. The content of the fermenter was constantly stirred at 300 ppm, the temperature was maintained at 37° C. and a pH set point 5.5 was controlled by addition of 24% ammonia water. Fermentation was completed in 7 hours after that glucose was completely utilized by Lb. animalis CHCC10506. The fermentation broth was cooled down to 4° C. and processed by centrifugation at 4° C. A cell concentrate was prepared by 40× concentration of the fermentation broth.

The recipes for various additive compositions were adapted from the book Wolkers & Oldenhof (Eds), Cryopreservation and Freeze-drying Protocols, Third edition (2015) Chapter 24: Freeze-Drying of Lactic Acid Bacteria, Fernanda Fonseca, Stéphanie Cenard, and Stéphanie Passot, p. 480, in which following additive composition was given: 200 g/I sucrose, 9 g/I NaCl and 5 g/I Na-ascorbate in demineralized water. For the purpose of preparing additives with different test compositions, the basic concentration of the NaCl was kept at 0.9% (w/w) and the concentration of Na-ascorbate was 0.5% (w/w) in the final additive, while sucrose was replaced with other carbohydrates or sugar alcohol. The following carbohydrates were tested in the additive: 20% (w/w) glucose, 20% (w/w) lactose, 20% (w/w) FOS, 20% (w/w) Glucidex® IT 12, 20% (w/w) trehalose and 20% gum arabic.

Due to the limited solubility of inositol in water (max 14%), the inositol additive was prepared as 10% (w/w) inositol in the final additive solution.

Pectin also has limited solubility in water and therefore pectin additive was prepared as 2% pectin (w/w) in the final additive solution.

When desired, combinations of carbohydrates in the final additive were applied, with a total sum of 20% (w/w) carbohydrates in the final additive.

The various single additives were sourced as follows: glucose (dextrose monohydrate, Roquette Freres, France), lactose (lactose monohydrate, Arla Food Ingredients Group P/S, Denmark), Glucidex® IT12 (trade name of maltodextrin DE 12, Roquette Freres, France), fructooligosaccharides (FOS, Fructo-oligosaccharide 950P, Beghin-Meiji, France), trehalose (trehalose dihydrate, Cargill, Germany), inositol (Zhucheng, Haotian Pharmaceutical Co., Ltd., China), GENU® pectin YM-115-H (CP Kelco, Denmark) and gum arabic (Willy Benecke GmbH, Natural Gums, Germany). FOS is a mixture of saccharides with chain length varying between one and five saccharide units, 31-43 g GF 2/100 g; 47-59 g GF3/100G and 4-16 g GF4/100 g FOS. Glucidex® IT 12 contains oligomers with 11-14 dextrose equivalents (97%); glucose (1%) and disaccharide (2%).

Carbohydrates or sugar alcohols were autoclaved for 20 min at 121° C.; pectin was pasteurized for 10 minutes at 80° C. Sodium ascorbate was prepared by sterile-filtration and mixed with autoclaved carbohydrates/sugar alcohol immediately before cryoformulation.

Aliquots of cell concentrate were transferred to 200 ml DASGIP parallel bioreactors (Eppendorf, Germany) and kept at predefined temperatures under nitrogen blanket. Constant stirring of cell concentrates at 100 rpm was applied. Formulation of cell concentrates by additives was done by addition of precooled additives to the cell concentrates. Cell concentrate was mixed with additive at a ratio 1:2 (w/w), i.e. 1 g of cell concentrate and 2 g of additive. The formulated concentrates were incubated for predefined time periods, i.e. holding times, from 15 minutes to 8 hours. pH was measured from the start to the end of the duration of the holding time.

After termination of the cell conditioning, formulated cell concentrates were pelletized in liquid nitrogen and stored in a freezer at −55° C. When production of freeze-dried granulate was desired, frozen pelletized concentrates were subjected to freeze-drying and freeze-dried granulates were produced.

Freeze-dried granulates were sealed in aluminium bags and subjected to accelerated stability studies at 37° C. for a period of up to 12 weeks.

Determination of cell counts by flowcytometry. Sample preparation and flowcytometric assay was carried out according to disclosure of Worm et al. in paragraphs [0059-0061] (EP 1 891 436 B1). Flowcytometry assay identifies total cells and active cells. Active cells are characterized by capability to maintain their cell membrane potential and their cell membrane is intact. The results are a good guide to the degree of preservation of the cells.

Enumeration of viable cells. Viable cell counts of Lactobacillus animalis CHCC10506 were determined in freeze-dried granulates sampled immediately after freeze-drying and at selected time points during the stability studies. Standard pour-plating method was used. The freeze-dried material was suspended in sterile peptone saline diluent and homogenized by stomaching. After 30 minutes of revitalization, stomaching was repeated and the cell suspension was serially diluted in peptone saline diluent. The dilutions were plated in duplicates on MRS agar (BD Difco™ Lactobacilli MRS Agar, Fisher Scientific). The agar plates were incubated anaerobically for three days at 37° C. Plates with 30-300 colonies were chosen for counting of colony forming units (CFU). The result was reported as average CFU/g freeze-dried sample, calculated from the duplicates.

Stability of cells was assessed from the difference between CFU/g measured at the time 0 of the stability trials and at the specific sampling points of the stability test period. Loss of viability was quantified as CFU log loss.

Bacterial cell surface characteristics, such as bacterial cell surface hydrophobicity and zeta potential, were determined for cells from frozen crude cell concentrate and for cells from freeze-dried products.

Cell surface hydrophobicity was measured by the MATH method, and interfacial adhesion curves were determined. The method of Schär-Zammaretti & Ubbink ((2003) Biophysical Journal 85, 4076-4092)) was applied with modified buffer strength and the use of a cell wash in the initial step of the procedure: 1 g of cell concentrate or 0.2 g of freeze-dried granulate was resuspended in 10 ml of 100 mM sodium phosphate buffer (pH 7.0). The cell suspension was centrifuged at 5000 g for 10 minutes at a temperature ≤10° C. Supernatant was removed and cells were washed twice with the 100 mM sodium phosphate buffer. The washed cell pellet was resuspended in the 100 mM sodium phosphate buffer to optical density OD₆₀₀ nm of 0.5±0.05. The suspension was mixed and aliquots of 3 ml were pipetted into plastic tubes. Hexadecane (99% purity, Sigma Aldrich) was added to the cell suspension in the following volumes: 10 μl, 30 μl, 100 μl, 200 μl, 400 μl, 800 μl, 1400 μl and 2000 μl hexadecane. Each combination of hexadecane and cell suspension in the buffer, Φ[V_{H}/V_{B}], was prepared in triplicate. The tubes were closed and the mixtures were vortexed one by one for 30 seconds at highest speed. Vortexing was repeated for 30 seconds once again for the whole sample series. The samples were left to rest for 5 minutes. 2 ml of aqueous phase was transferred to a cuvette for measurement of the OD₆₀₀ nm. Bacterial cell surface hydrophobicity (BCSH) was calculated from the fraction of bacteria which adhered to the hexadecane/water interface according to the formula:

BCSH (%)=[(Initial OD₆₀₀−Final OD₆₀₀)/Initial OD₆₀₀]*100

A cell surface is classified as non-hydrophobic, i.e. hydrophilic, if partitioning of cells gives BCSH <20%. A hydrophobic cell surface is characterized by partitioning of cells with BCSH >50%, and a moderately hydrophobic surface has a BCSH in the range 20-50% (Lee and Yii (1996) Letters in Applied Microbiology 23: 343-346).

Zeta potential measurement. 1 g of cell concentrate or 0.2 g of freeze-dried granulate was suspended in 1 mM NaCl and washed twice by centrifugation at 5000 g for 10 minutes at 10° C. The washed cell pellet was resuspended in 1 mM NaCl to OD_{600 nm}=0.5±0.05. Aliquots of the cell suspension were pH adjusted with 20 mM HCl or 20 mM NaOH to create a serial of samples within the pH range 2.70 to 8.45. The Zeta potential of samples was measured in apparatus SZ-100Z (Horiba Scientific, France). Measurement was done in triplicate for each pH. The results are presented as average of triplicates with standard deviation. The pH at which the Zeta potential becomes zero is called the isoelectric point (pI).

### Example 1—Characterization of Lactobacillus animalis Cell Concentrate

Four batches of Lactobacillus animalis CHCC10506 cell concentrate were produced in four fermentation trials. Microscopy of the cell concentrates revealed that cells appeared as single rods and in pairs. The average composition of the cell concentrate is presented in Table 1.

TABLE 1
Composition of cell concentrate (n = 4)

| Parameter | Value |
|---|---|
| pH | 5.5 ± 0.1 |
| Dry matter (%) | 15.9 ± 0.4 |
| Total cells/g | 3.53E+11 ± 0.04E+11 |
| Active cells/g | 3.34E+11 ± 0.07E+11 |
| Active cells/Total cells (%) | 94.7 ± 2.4 |

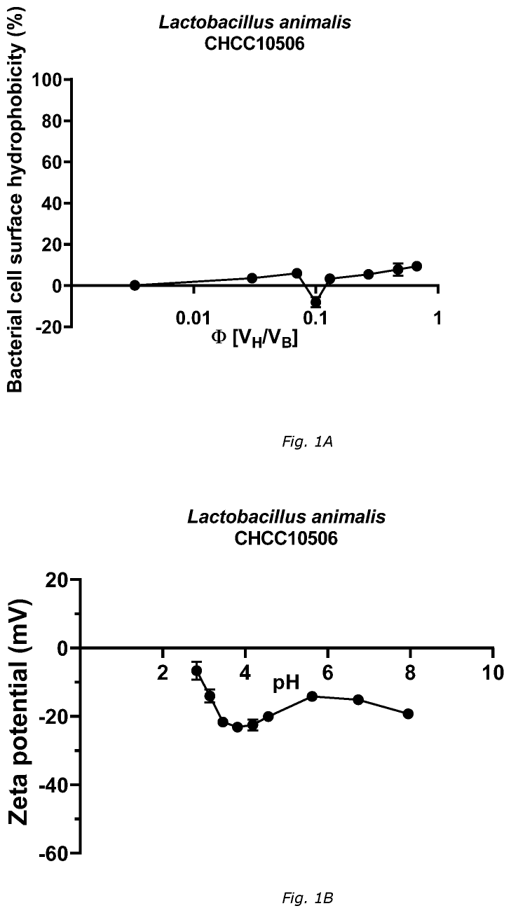

An interfacial adhesion curve of the crude cell concentrate is presented in FIG. 1A. The shape of curve reflected a very poor partitioning of cells towards the hexadecane phase despite the increase in the volume of hexadecane in the mix with the bacterial suspension. The complete lack of a sigmoidal shape of the curve and bacterial adhesion parameter BCSH below 20% demonstrated the hydrophilic nature of the cell surface. Measurement of the Zeta potential in FIG. 1B revealed that the cell surface is negatively charged, with a pI<below pH 2.82 and with a maximum Zeta potential of −23.1±0.2 mV.

### Example 2—Cell Response to Additive Containing Single Carbohydrate or Polyol and Stability of Freeze-Dried Cells

Aliquots of Lactobacillus animalis CHCC10506 concentrate were formulated with additives containing the selected single carbohydrate. The composition of the additives and formulated cell concentrates is shown in Table 2.

TABLE 2
Composition of additives and formulated cell concentrates

| | | | | | Formulated cell |
|---|---|---|---|---|---|
| | Additive | | Cell concentrate | | concentrate |
| | Composition | Amount | Composition | Amount | Composition |
| No. | (% dry matter) | (g) | (% dry matter) | (g) | (% dry matter) |
| 1 | 20% Glucose | 66 | 15.9% | 33 | 13.3% Glucose |
| | 0.9% NaCl | | | | 0.6% NaCl |
| | 0.5% Na-ascorbate | | | | 0.3% Na-ascorbate |
| | | | | | 5.3% CC* |
| 2 | 20% lactose | 66 | 15.9% | 33 | 13.3% Lactose |
| | 0.9% NaCl | | | | 0.6% NaCl |
| | 0.5% Na-ascorbate | | | | 0.3% Na-ascorbate |
| | | | | | 5.3% CC |
| 3 | 20% Glucidex ® IT 12 | 66 | 15.9% | 33 | 13.3% Glucidex ® IT 12 |
| | 0.9% NaCl | | | | 0.6% NaCl |
| | 0.5% Na-ascorbate | | | | 0.3% Na-ascorbate |
| | | | | | 5.3% CC |
| 4 | 20% FOS | 66 | 15.9% | 33 | 13.3% FOS |
| | 0.9% NaCl | | | | 0.6% NaCl |
| | 0.5% Na-ascorbate | | | | 0.3% Na-ascorbate |
| | | | | | 5.3% CC |
| 5 | 20% Trehalose | 66 | 15.9% | 33 | 13.3% Trehalose |
| | 0.9% NaCl | | | | 0.6% NaCl |
| | 0.5% Na-ascorbate | | | | 0.3% Na-ascorbate |
| | | | | | 5.3% CC |
| 6 | 10% Inositol | 66 | 15.9% | 33 | 6.7% Inositol |
| | 0.9% NaCl | | | | 0.6% NaCl |
| | 0.5% Na-ascorbate | | | | 0.3% Na-ascorbate |
| | | | | | 5.3% CC |
| 7 | 2% GENU-Pectin | 66 | 15.9% | 33 | 1.3% GENU Pectin |
| | 0.9% NaCl | | | | 0.6% NaCl |
| | 0.5% Na-ascorbate | | | | 0.3% Na-ascorbate |
| | | | | | 5.3% CC |
| 8 | 20% Gum arabic | 72 | 15.9% | 36 | 13.3% Gum arabic |
| | 0.9% NaCl | | | | 0.6% NaCl |
| | 0.5% Na-ascorbate | | | | 0.3% Na-ascorbate |
| | | | | | 5.3% CC |

*CC = cell concentrate
*FOS = fructooligosaccharides
** Glucidex ® IT 12 is a trade name for maltodextrin DE 12.

Formulated cell concentrates were held for 2 hours at 10° C. The pH was measured and the difference between the start and the end of the holding time was calculated (Table 3).

TABLE 3
Change of pH in formulated cell concentrates after 2 h at 10° C.

| Formulated cell | pH | pH | ΔpH |
|---|---|---|---|
| concentrate | start | end | (start to end) |
| 1 (glucose) | 5.47 | 4.55 | 1.02 |
| 2 (lactose) | 5.54 | 5.06 | 0.48 |
| 3 (maltodextrin) | 5.50 | 5.12 | 0.38 |
| 4 (FOS) | 5.47 | 4.53 | 0.94 |
| 5 (trehalose) | 5.59 | 5.36 | 0.23 |
| 6 (inositol) | 5.54 | 5.60 | −0.06 |
| 7 (pectin) | 5.54 | 5.16 | 0.38 |
| 8 (gum arabic) | 5.65 | 5.63 | 0.02 |

It was clear that the majority of additives energized and activated cells in formulated cell concentrates. Activation of metabolism by additives 1, 2, 3, 4, 5 and 7, i.e. glucose, lactose, maltodextrin, FOS, trehalose and pectin, resulted in production of acid, whereby pH was reduced by 0.23-1.02 pH unit. In contrast, it can be seen that additive 6 with inositol and additive 8 with gum arabic did not lead to an appreciable drop in pH, indicating that acids were not produced.

Activation of cells during the 2 hours of holding time at 10° C. was not found to be correlated to the cell growth in concentrate. After termination of the holding time and freezing of formulated cell concentrates, cell counts were determined by flowcytometry (Table 4). The result was as expected when dilution of crude cell concentrate by additive and cell morphology was taken into consideration. The share of active cells differentiated substantially, showing that activation of metabolism by specific additive had a different effect on the capability of cells to survive the freezing. Additives 3, 4, 5 and 7 were found to have a better cryoprotective effect than additives 1, 2, 6 and 8.

TABLE 4
Cell counts in frozen formulated cell concentrates. Results
are presented as averages with coefficient of variation.

| Formulated cell | | (Active cells/g)/ |
|---|---|---|
| concentrate no. | Total cells/g | (Total cells/g) (%) |

| | | | |
|---|---|---|---|
| 1 (glucose) | 1.43E+11 | (9.9%) | 41 |
| 2 (lactose) | 1.27E+11 | (3.2%) | 43 |
| 3 (maltodextrin) | 1.25E+11 | (6.0%) | 71 |
| 4 (FOS) | 1.21E+11 | (2.7%) | 78 |
| 5 (trehalose) | 1.21E+11 | (7.7%) | 72 |
| 6 (inositol) | 1.22E+11 | (6.0%) | 42 |
| 7 (pectin) | 1.24E+11 | (4.8%) | 68 |
| 8 (gum arabic) | 1.25E+11 | (13%) | 37 |

Formulated cell concentrates were processed to freeze-dried granulates and viable cells were determined in freeze-dried materials by CFU analysis (Table 5).

TABLE 5
CFU counts in freeze-dried granulates

| Freeze-dried granulate | CFU/g |
|---|---|
| 1 (glucose) | 1.9E+11 |
| 2 (lactose) | 2.6E+11 |
| 3 (maltodextrin) | 2.0E+11 |
| 4 (FOS) | 2.8E+11 |
| 5 (trehalose) | 2.5E+11 |
| 6 (inositol) | 3.1E+11 |
| 7 (pectin) | 4.1E+11 |
| 8 (gum arabic) | 8.3E+10 |

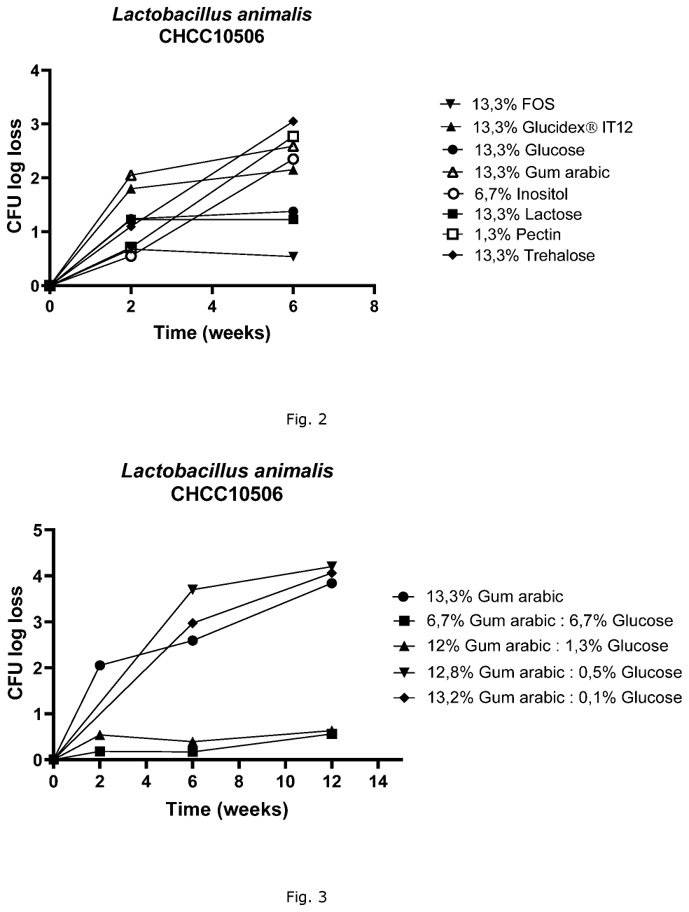

The freeze-dried granulates were subjected to accelerated stability test at 37° C. for a period of 6 weeks. Viability of cells was monitored by CFU analysis and loss of CFU is depicted in FIG. 2. The lower the CFU loss, the better the performance. The lowest loss of viable cells were found in formulations 1, 2 and 4, i.e. formulations with glucose-, lactose- and FOS-containing additive, showing 1.4; 1.2 and 0.5 CFU log loss after 6 weeks of accelerated stability trial.

### Example 3—Cell Response to Additive Containing Mixture of Glucose and Gum Arabic and Stability of Freeze-Dried Cells

Cell concentrates of Lactobacillus animalis CHCC10506 were admixed with protective additives containing a combination of fermentable sugar and a non-fermentable polysaccharide such as glucose and gum arabic, respectively. Gum arabic was combined at different ratios with glucose, to provide a combined carbohydrate concentration 20%, or with 20% gum arabic alone for comparison. Composition of the additives and formulated cell concentrates are shown in Table 6.

TABLE 6
Composition of the additives and formulated cell concentrates

| | | | | | Formulated cell |
|---|---|---|---|---|---|
| | Additive | | Cell concentrate | | concentrate |
| | Composition | Amount | Composition | Amount | Composition |
| No. | (% dry matter) | (g) | (% dry matter) | (g) | (% dry matter) |
| 8 | 20% Gum arabic | 72 | 15.9% | 36 | 13.3% Gum arabic |
| | 0.9% NaCl | | | | 0.6% NaCl |
| | 0.5% Na-ascorbate | | | | 0.3% Na-ascorbate |
| | | | | | 5.3% Cell concentrate |
| 9 | 10% Gum arabic | 72 | 15.9% | 36 | 6.7% Gum arabic |
| | 10% Glucose | | | | 6.7% Glucose |
| | 0.9% NaCl | | | | 0.6% NaCl |
| | 0.5% Na-ascorbate | | | | 0.3% Na-ascorbate |
| | | | | | 5.3% Cell concentrate |
| 10 | 18% Gum arabic | 72 | 15.9% | 36 | 12% Gum arabic |
| | 2% Glucose | | | | 1.3% Glucose |
| | 0.9% NaCl | | | | 0.6% NaCl |
| | 0.5% Na-ascorbate | | | | 0.3% Na-ascorbate |
| | | | | | 5.3% Cell concentrate |
| 11 | 19.2% Gum arabic | 72 | 15.9% | 36 | 12.8% Gum arabic |
| | 0.8% Glucose | | | | 0.5% Glucose |
| | 0.9% NaCl | | | | 0.6% NaCl |
| | 0.5% Na-ascorbate | | | | 0.3% Na-ascorbate |
| | | | | | 5.3% Cell concentrate |
| 12 | 19.8 Gum arabic | 72 | 15.9% | 36 | 13.2% Gum arabic |
| | 0.2% Glucose | | | | 0.1% Glucose |
| | 0.9% NaCl | | | | 0.6% NaCl |
| | 0.5% Na-ascorbate | | | | 0.3% Na-ascorbate |
| | | | | | 5.3% Cell concentrate |

Formulated cell concentrates were conditioned for 2 hours at 10° C. and pH was monitored (Table 7). All formulations with glucose-containing additives, 9-12, exhibited acid production and concomitant pH reduction, while formulation 8 with gum arabic alone did not show any acidification.

TABLE 7
Change of pH in formulated cell concentrates
after 2 hours at 10° C.

| Formulated cell | pH | pH | ΔpH |
|---|---|---|---|
| concentrate | start | end | (start to end) |
| 8 | 5.65 | 5.63 | 0.02 |
| 9 | 5.54 | 4.50 | 1.04 |
| 10 | 5.61 | 4.52 | 1.09 |
| 11 | 5.63 | 4.58 | 1.05 |
| 12 | 5.65 | 4.94 | 0.71 |

Frozen formulated cell concentrates made at the end of holding time were analysed by flowcytometry (Table 8). Potencies of the frozen products were as expected when dilution of crude concentrate by additives and morphology was taken into consideration. This finding confirmed that cells were not growing under conditions tested.

TABLE 8
Cell counts in formulated cell concentrates. Results are presented
as averages (n = 4) with coefficient of variation in parentheses.

| Formulated cell concentrate | Total cells/g | |
|---|---|---|
| 8 | 1.25E+11 | (13.0%) |
| 9 | 1.42E+11 | (11.1%) |
| 10 | 1.14E+11 | (7.0%) |
| 11 | 1.10E+11 | (6.8%) |
| 12 | 1.05E+11 | (4.0%) |

Frozen, formulated cell concentrates were freeze-dried and viability of cells in freeze-dried granulates was determined by CFU method (Table 9). Survival of freeze-drying was best when cell concentrate was formulated with additive 9, containing 10% gum arabic and 10% glucose as carbohydrates.

TABLE 9
CFU counts in freeze-dried granulates

| Freeze-dried granulate | CFU/g |
|---|---|
| 8 | 8.3E+10 |
| 9 | 2.0E+11 |
| 10 | 1.1E+11 |
| 11 | 1.0E+11 |
| 12 | 7.7E+10 |

FIG. 3 shows the log loss of viable cells in freeze-dried granulates 8-12, measured by CFU method, after 0, 2, 6 and 12 weeks storage of the freeze-dried product at 37° C. in sealed aluminium foil bags.

Bearing in mind that the parameter that is being measured is loss of CFU, the additive with a mixture of 10% gum arabic+10% glucose was particularly effective, and a mixture of 18% gum arabic+2% glucose was nearly as good. It can also be seen that there needs to be enough of the fermentable saccharide to energise and activate the cells sufficiently; using 0.2% or 0.8% glucose in place of 0.2% or 0.8% gum arabic, respectively, actually led to an antagonistic effect, even though glucose on its own was better than gum arabic on its own. The replacement of 2% or 10% of the gum arabic with glucose, however, led to a synergistic effect, which was surprising.

### Example 4—Cell Response to a Mixture of Activating and Non-Activating Carbohydrates with Different Molecule Length and Stability of Freeze-Dried Cells

The length of the activating molecule was explored. The method of Example 3 was repeated using in additives as carbohydrates 10% gum arabic mixed with 10% lactose, 10% fructooligosaccharides (FOS), 10% Glucidex® IT 12 (i.e. maltodextrin DE 12) or 10% inositol, and compared with the 10% gum arabic plus 10% glucose mixture that was used in Example 3. Compositions of the additives and formulated cell concentrates are presented in Table 10.

TABLE 10
Composition of the additives and formulated cell concentrates

| | | | | | Formulated cell |
|---|---|---|---|---|---|
| | Additive | | Cell concentrate | | concentrate |
| | Composition | Amount | Composition | Amount | Composition |
| No. | (% dry matter) | (g) | (% dry matter) | (g) | (% dry matter) |
| 9 | 10% Gum arabic | 72 | 15.9% | 36 | 6.7% Gum arabic |
| | 10% Glucose | | | | 6.7% Glucose |
| | 0.9% NaCl | | | | 0.6% NaCl |
| | 0.5% Na-ascorbate | | | | 0.3% Na-ascorbate |
| | | | | | 5.3% CC |
| 13 | 10% Gum arabic | 72 | 15.9% | 36 | 6.7% Gum arabic |
| | 10% Lactose | | | | 6.7% Lactose |
| | 0.9% NaCl | | | | 0.6% NaCl |
| | 0.5% Na-ascorbate | | | | 0.3% Na-ascorbate |
| | | | | | 5.3% CC |
| 14 | 10% Gum arabic | 72 | 15.9% | 36 | 6.7% Gum arabic |
| | 10% FOS | | | | 6.7% FOS |
| | 0.9% NaCl | | | | 0.6% NaCl |
| | 0.5% Na-ascorbate | | | | 0.3% Na-ascorbate |
| | | | | | 5.3% CC |
| 15 | 10% Gum arabic | 72 | 15.9% | 36 | 6.7% Gum arabic |
| | 10% Glucidex ® IT 12 | | | | 6.7% Glucidex ® IT 12 |
| | 0.9% NaCl | | | | 0.6% NaCl |
| | 0.5% Na-ascorbate | | | | 0.3% Na-ascorbate |
| | | | | | 5.3% CC |
| 16 | 10% Gum arabic | 70 | 15.9% | 35 | 6.7% Gum arabic |
| | 10% Inositol | | | | 6.7% Inositol |
| | 0.9% NaCl | | | | 0.6% NaCl |
| | 0.5% Na-ascorbate | | | | 0.3% Na-ascorbate |
| | | | | | 5.3% CC |

Formulated cell concentrates were kept at 10° C. for 2 hours and change of pH during this holding time was monitored (Table 11). Production of acids with concomitant pH reduction was observed in concentrate formulations with additives containing carbohydrates, but not with inositol. Highest activation of metabolism and acidification was obtained with additives 9, 13 and 14, which contained shorter carbohydrate molecules such as glucose, lactose and fructooligosaccharides, respectively. Acid production from oligomers of Glucidex® IT 12 was limited.

TABLE 11
Change of pH in formulated cell concentrates
after 2 hours at 10° C.

| Formulated cell | pH | pH | ΔpH |
|---|---|---|---|
| concentrate | start | end | (start to end) |
| 9 | 5.54 | 4.50 | 1.04 |
| 13 | 5.65 | 4.91 | 0.74 |
| 14 | 5.58 | 4.52 | 1.06 |
| 15 | 5.61 | 5.26 | 0.35 |
| 16 | 5.59 | 5.61 | −0.02 |

The Total cell counts/g were measured in the frozen products prior to freeze-drying (Table 12). Total cell counts/g frozen materials within this group were very similar and the values were as expected for the scenario without cell growth.

TABLE 12
Total cell counts in frozen formulated cell concentrates.
Results are presented as averages (n = 4)
with coefficient of variation in parentheses.

| Formulated cell concentrate | Total cells/g | |
|---|---|---|
| 9 | 1.42E+11 | (11.1%) |
| 13 | 1.25E+11 | (8.9%) |
| 14 | 1.25E+11 | (17.8%) |
| 15 | 1.27E+11 | (7.2%) |
| 16 | 1.18E+11 | (1.9%) |

Viability of cells in freeze-dried products was determined and results of CFU analysis are summarized in Table 13. Best survival of freeze-drying was obtained in formulation with a mixture of gum arabic and inositol (16), followed by formulations with a mixture of gum arabic additive and shorter carbohydrates, i.e. glucose (9); lactose (13) and FOS (14). Lowest viability of cells was measured in freeze-dried product formulated with a mixture of gum arabic and oligomers of Glucidex® IT 12 (additive 15).

TABLE 13
CFU counts in freeze-dried granulates

| Freeze-dried granulate | CFU/g |
|---|---|
| 9 | 2.0E+11 |
| 13 | 1.6E+11 |
| 14 | 3.3E+11 |
| 15 | 9.9E+10 |
| 16 | 5.2E+11 |

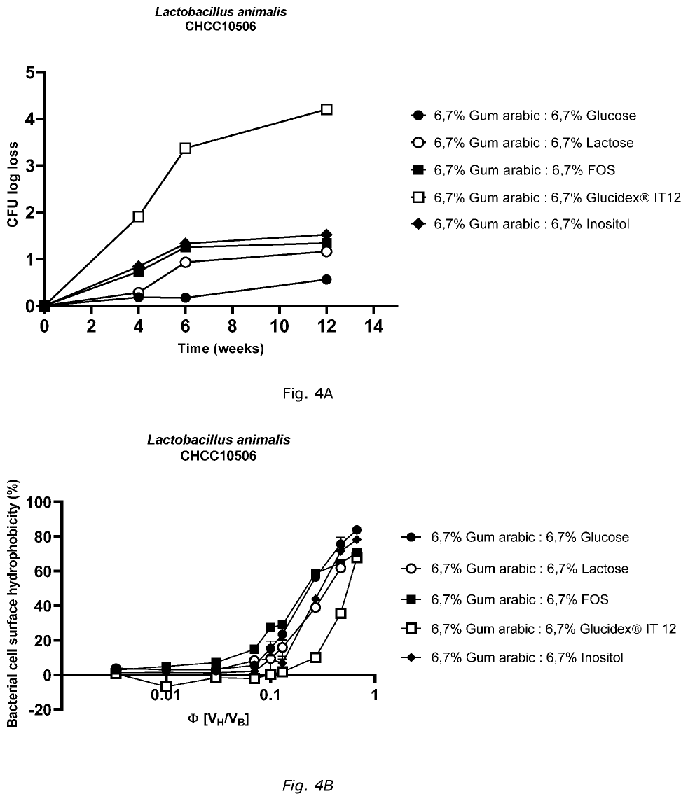

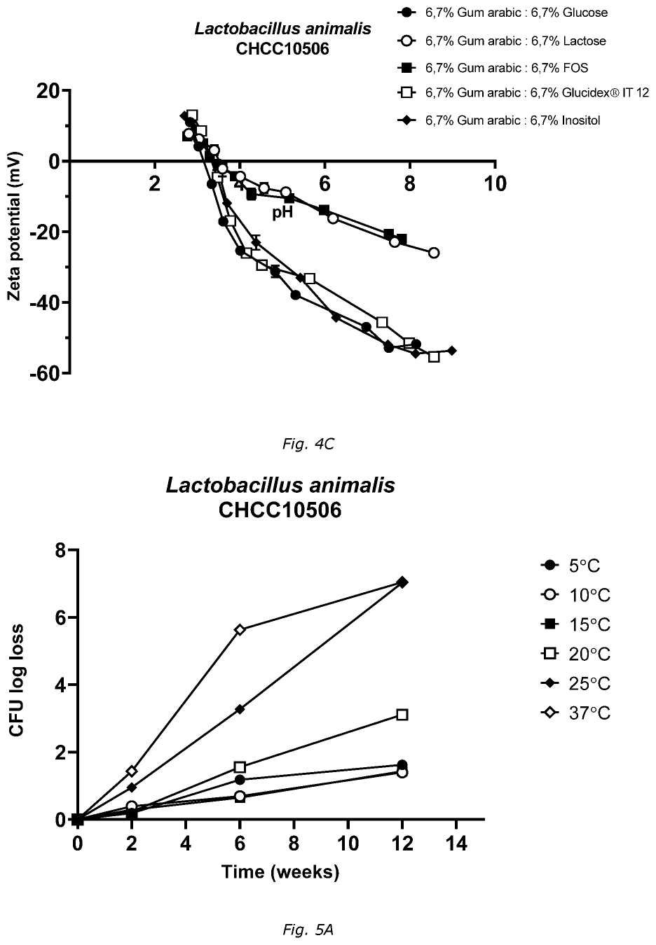

The freeze-dried products were packed in aluminium pouches and subjected to stability test at 37° C. for 12 weeks. The CFU/g were measured at 0, 2, 6 and 12 weeks. From results in FIG. 4A it can be seen that, in general, the protective effect was related to the length of the molecule of compound that was mixed with the non-fermentable gum arabic. The best stability was obtained with the monosaccharide glucose as additional carbohydrate. Next best was the disaccharide lactose and FOS with short carbohydrate chains of three to five sugar molecules. Maltodextrin Glucidex® IT 12, primarily composed of oligomers with 11-14 DE (which is again a mixture, with the chain length varying between one and 17 saccharide units) gave the poorest stability of the freeze-dried product. Hence, the shorter the carbohydrate combined with gum arabic, the better the stability of Lactobacillus animalis CHCC10506.

The polyol inositol, used in addition to gum arabic, showed surprisingly good protection of cells in the stability test despite of the anomaly in the acidification pattern during the formulation of concentrate. The CFU loss with inositol—gum arabic additive was 1.52 log units after 12 weeks incubation at 37° C., what was very close to the performance of FOS—gum arabic matrix, showing a CFU loss of 1.34 log unit under the same conditions. Nevertheless, the mixture of gum arabic and inositol performed better in the stability test than the combination of partially fermentable carbohydrate Glucidex® IT 12 and gum arabic or the completely non-fermentable gum arabic alone from Example 2, giving at the end of stability test 3.84 CFU log loss. The inositol—gum arabic matrix was thus still showing the surprising effect.

Interfacial adhesion curves of cells from freeze-dried products produced for Example 4 are shown in FIG. 4B. In comparison to the flat profile of crude cell concentrate, the curves acquired a more sigmoidal shape what correlated with modulation of cell surface and increasing of the cell surface hydrophobicity. There was a clear difference in partitioning of cells in the assay and in the shape of interfacial adhesion curves, when cells were formulated with a combination of gum arabic and the partially fermentable carbohydrate Glucidex® IT 12 versus the group of cells formulated with a combination of gum arabic and readily fermentable carbohydrates (glucose, lactose, FOS). Inclusion of readily fermentable carbohydrates in the additive and conditioning of cell concentrates for 2 hours at 10° C. modified the cell surface substantially. The overall character of the cell surface turned towards hydrophobicity. Partitioning of cells to hexadecane/water interface and bringing the BCSH value above 20% was evident in these formulations when hexadecane/buffer ratio Φ[V_{H}/V_{B}], became ≥0.10-0.27. Conditioning of cells with a mixture of gum arabic and Glucidex® IT 12 modified also the cell surface. However, the hydrophobic patches on the cell surface were less abundant because partitioning above BCSH of 20% was achieved only for Φ≥0.47.

Despite of the lack of the evidence that inositol was metabolised, the MATH assay revealed a significant change in the profile of interfacial adhesion curve for freeze-dried cells formulated with the additive containing a mixture of inositol and gum arabic. The interfacial adhesion curve, and especially the region for Φ>0.13, with a steep increase of BCSH, showed that the cells surface acquired a pattern indicating presence of hydrophobic compounds.

Measurement of Zeta potential of cells from freeze-dried products produced for Example 4 is presented in FIG. 4C. The Zeta potential profiles are differentiating products into two groups. The first product group comprised cells, which have been conditioned in a mixture of gum arabic and disaccharide lactose (formulation 13) or gum arabic with FOS (formulation 14), i.e. in formulations with fermentable saccharides of heterogenous nature. The surfaces of these cells were negatively charged with a maximum Zeta potential of −22.9 mV (pH 8.56) and −20.6 mV (pH 7.8), for lactose and FOS-conditioned cells, respectively. The isoelectric points were found to be 3.51 and 3.42 for lactose and FOS-conditioned cells, respectively. In comparison to characteristics of cells in crude concentrate, cell surface charges for this first group was similar to the cells in crude cell concentrate, but the isoelectric points were at least 0.6 pH units higher than that of the cell concentrate. The second group of Zeta potential profiles comprised cells conditioned in a mixture of gum arabic and glucose (formulation 9), or gum arabic plus Glucidex® IT 12 (formulation 15), or gum arabic+inositol (formulation 16). The nature of fermentable saccharide in this second group was homogenous, either based on the glucose unit (glucose and Glucidex® IT 12) or inositol. These cells had much more negative charge on cell surface, with a maximum Zeta potential of −51.8 mV (pH 8.14), −55.4 (pH 8.56) mV and −54.5 mV (pH 8.13) and isoelectric points 3.15; 3.34 and 3.41; for cells conditioned with glucose, Glucidex® IT 12 and inositol-containing additive, respectively. In comparison to crude cell concentrate the overall cell surfaces of this second group was much more negatively charged and the isoelectric point was more than 0.32 pH unit higher.

When compiling outcomes of the activation step, MATH assay and Zeta potential assay and the freeze-drying process, all these findings together provide evidence that freeze-dried Lb. animalis CHCC10506, made by pre-processing at 10° C. with a mixture of readily fermentable carbohydrates or inositol and the non-fermentable polysaccharide, acquired modified cell surface with increased degree of hydrophobicity. Increased cell surface hydrophobicities were in the stability tests positively correlated with enhanced viability of cells in freeze-dried products stored under unfavourable conditions.

### Example 5—Effect of Temperature

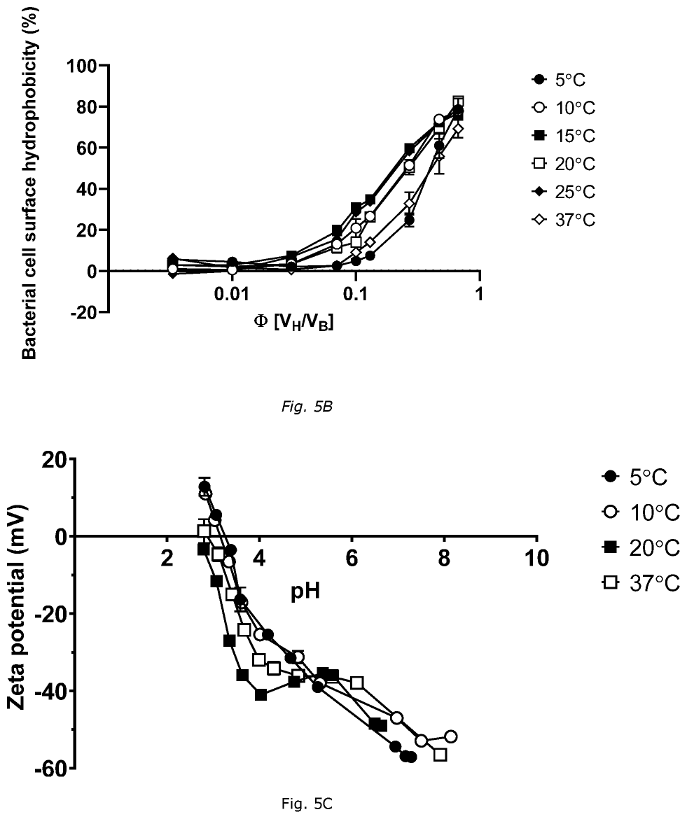

It is believed that the fermentable carbohydrate energises the cells during conditioning before freezing. A formulation of cell concentrate of Lactobacillus animalis CHCC 10506, was made by mixing 35 g of cell concentrate and 70 g of glucose-gum arabic containing additive 9, specified in Example 3. The formulated cell concentrate was held for 2 hours at different temperatures from the interval 5° C. to 37° C. to explore the effect of temperature. The following parameters were measured: increase in acid production by pH decrease across the 2 h period (shown in Table 14 below); quantification of Total cells/g frozen product when the frozen product was made from formulated cell concentrate after 2 hours of holding time (Table 15) and cell viability in freeze-dried product immediately after drying (Table 16); and the stability when the freeze-dried granulated product was stored at 37° C. in aluminium pouches (FIG. 5A).

From the pH measurement it can be seen there was already a small change in pH at 5° C., which indicated that activation of metabolism and acid production was initiated already at this temperature. The change in pH increased as the temperature during concentrate conditioning was increased to 10° C., 15° C., 20° C., 25° C. and 37° C.

TABLE 14
pH change in formulated cell concentrates after 2 h holding
time at temperatures from 5° C. to 37° C.

| | | pH | pH | |
|---|---|---|---|---|
| Formulated cell | Temperature | Start of | End of | |
| concentrate | (° C.) | holding time | holding time | Δ pH |
| 17 | 5 | 5.52 | 5.15 | 0.37 |
| 18 | 10 | 5.60 | 4.54 | 1.06 |
| 19 | 15 | 5.60 | 4.18 | 1.42 |
| 20 | 20 | 5.60 | 3.95 | 1.65 |
| 21 | 25 | 5.58 | 3.82 | 1.76 |
| 22 | 37 | 5.70 | 3.77 | 1.93 |

Quantification of Total cell counts/g frozen materials revealed that after 2 hours of cell conditioning with the additive and freezing, the cell counts within the group were comparable. In comparison to the Total cells/g crude concentrate and with consideration of the dilution effect by the additive, it can be concluded that no growth of cells occurred during 2 hours of cell conditioning with the glucose-gum arabic additive even though the temperature was raised to 37° C., i.e. to the optimum growth temperature of the strain.

TABLE 15
Total cell counts in frozen formulated cell concentrates.
Results are presented as averages (n = 4)
with coefficient of variation in parentheses.

| Formulated cell concentrate | Total cells/g | |
|---|---|---|
| 17 | 1.20E+11 | (7%) |
| 18 | 9.27E+10 | (22.2%) |
| 19 | 1.1E+11 | (3.2%) |
| 20 | 1.09E+11 | (1.7%) |
| 21 | 1.09E+11 | (4.5%) |
| 22 | 9.94E+10 | (2.6%) |

CFU analysis of freeze-dried granules showed a better survival of freeze-drying process when formulated cell concentrate was conditioned at temperatures 5° C.-20° C., with temperature of 10° C. as the most beneficial conditioning temperature for potency of the freeze-dried product.

TABLE 16
CFU counts in freeze-dried granulates

| Freeze-dried granulate | CFU/g |
|---|---|
| 17 | 2.04E+11 |
| 18 | 3.53E+11 |
| 19 | 2.52E+11 |
| 20 | 1.59E+11 |
| 21 | 1.07E+11 |
| 22 | 8.49E+10 |

In terms of storage stability, it can be seen from FIG. 5A that the optimum temperature for cell conditioning in concentrate with the glucose-gum arabic additive was in range 5-15° C. (formulations 17-19). The CFU loss in these three freeze-dried products after 12 weeks storage at 37° C. were in range 1.4-1.6 log unit. Increase of the conditioning temperature to 20° C. resulted in deteriorated stability of freeze-dried product 20, with a final CFU loss of 3.1 log units. Conditioning the formulated cell concentrate at temperatures of 30° C. and 37° C. was correlated with a very poor stability of freeze-dried products 21 and 22, with the final CFU loss of 7 log units. We surmise that, although an increased temperature may lead to increased metabolism of the fermentable carbohydrate. However, activation of the metabolism of non-growing cells above certain temperature limit (in this case >20° C.) was found to reduce cell robustness during industrial processing and be detrimental for viability of cells in freeze-dried products exposed to non-favourable storage conditions.

Interfacial adhesion curves were made with freeze-dried products sampled immediately after freeze-drying. The results are shown in FIG. 5B. From the shape of the curves it was obvious that modulation of cell surface towards enhanced hydrophobicity occurred in the whole temperature interval from 5° C.-37° C. Formation of hydrophobic compounds on the cell surface was promoted by conditioning of cells in formulated cell concentrate at temperatures from 10° C.-25° C. In this case, partitioning of cells to hexadecane/water interface gave BCSH values higher than 20% for hexadecane/buffer ratio Φ≥0.10-0.13. When the cells were conditioned at the boundaries of the temperature interval investigated, i.e. at 5° C. and 37° C., the BCSH values became higher than 20% only for hexadecane/buffer ratio Φ≥0.27. This finding can be explained by a lower abundance of hydrophobic patches on the surface of cells pre-processed at 5° C. or 37° C.

Effect of conditioning temperature was investigated in relation to the cell surface charge of freeze-dried cells (FIG. 5C). Conditioning temperatures 5° C., 10° C., 20° C. and 37° C. resulted in formation of cell surfaces with a very negative charge. The maximum Zeta potential was determined to be −57 mV (pH 7.28), −52 mV (pH 7.50), −49 mV (pH 6.63) and −56 mV (pH 7.9) for cells incubated at 5° C., 10° C., 20° C. and 37° C., respectively. The isoelectric points of cells conditioned at 20° C. and 37° C. were <2.79 and equal to 2.87, respectively. In contrary, cells conditioned at 5° C. and 10° C., had isoelectric point of 3.26 and 3.15. These finding indicated that conditioning of cells at lower temperatures contributed to formation of cell surface structures that raised the isoelectric point.

Activation of non-growing cells and stimulation of metabolism by fermentable carbohydrate occurred in a broad temperature range from 5° C. to 37° C. As a result of thereof, hydrophobic structures were formed on the cell surface and the cell surface charge was modified. If the process was facilitated by temperatures between 5-15° C., superior stability of cells of Lactobacillus animalis CHCC10506 in freeze-dried products was achieved.

### Example 6—Effect of Holding Time

Using mixture of 10% gum arabic plus 10% glucose or 10% gum arabic plus 10% Glucidex® IT 12 in the additive, cell concentrates of Lactobacillus animalis CHCC 10506 were formulated to compositions given in Table 17.

TABLE 17
Composition of additives and formulated cell concentrates

| | | | | | Formulated cell |
|---|---|---|---|---|---|
| | Additive | | Cell concentrate | | concentrate |
| | Composition | Amount | Composition | Amount | Composition |
| No | (% dry matter) | (g) | (% dry matter) | (g) | (% dry matter) |
| 23-26 | 10% Gum arabic | 70 | 15.9% | 35 | 6.7% Gum arabic |
| | 10% Glucose | | | | 6.7% Glucose |
| | 0.9% NaCl | | | | 0.6% NaCl |
| | 0.5% Na-ascorbate | | | | 0.3% Na-ascorbate |
| | | | | | 5.3% CC |
| 27-30 | 10% Gum arabic | 70 | 15.9% | 35 | 6.7% Gum arabic |
| | 10% Glucidex ® IT 12 | | | | 6.7% Glucidex ® IT 12 |
| | 0.9% NaCl | | | | 0.6% NaCl |
| | 0.5% Na-ascorbate | | | | 0.3% Na-ascorbate |
| | | | | | 5.3% CC |

Formulated cell concentrates were kept at 10° C. for varying time periods in range 15 minutes-8 hours. pH changes during the holding time were measured and the results are presented in Table 18. When conditioning of cells with both additive was restricted to 15 minutes, activation of metabolism and acid production were not evident (formulations 23 and 27). In contrary, when holding time was 2 hours and longer, presence of glucose in the additive showed a pronounced effect on activation of cell metabolism, formation of acids and concomitant pH reduction. The longer the holding time, the larger pH drop in formulated cell concentrate. Metabolism and acid production were also activated by Glucidex® IT12—containing additive. However, the extent of pH reduction with Glucidex® IT12—additive was smaller than with glucose-additive and without linear correlation between the duration of the holding time and pH drop. It was confirmed by HPLC analysis that the net production of lactic acid increased from 0 g/kg to 6 g/kg in glucose-formulated concentrate for 15 minutes and 8 h holding time, respectively. In Glucidex® IT12-formulated cell concentrate, the net lactic acid production increased from 0 g/kg to 1.7 g/kg for 15 minutes and 8 h holding time, respectively.

TABLE 18
Change of pH in formulated cell concentrates
at 10° C. after specified holding times

| | | pH | pH | |
|---|---|---|---|---|
| | | Start of | End of | |
| No. | Holding time | holding time | holding time | Δ pH |

| | | | | | |
|---|---|---|---|---|---|
| 23 | 15 | minutes | 5.53 | 5.54 | −0.01 |
| 24 | 2 | hours | 5.49 | 4.60 | 0.89 |
| 25 | 4 | hours | 5.54 | 4.23 | 1.31 |
| 26 | 8 | hours | 5.49 | 4.09 | 1.40 |
| 27 | 15 | minutes | 5.64 | 5.62 | 0.02 |
| 28 | 2 | hours | 5.58 | 5.26 | 0.32 |
| 29 | 4 | hours | 5.49 | 5.15 | 0.34 |
| 30 | 8 | hours | 5.54 | 4.88 | 0.66 |

Frozen formulated cell concentrates, made after termination of cell conditioning, were analysed by flowcytometry (Table 19). The total cell counts of frozen products were as expected when dilution of crude concentrate by additives was taken into consideration. The Total cell counts/g in formulations 23-26 with the glucose—gum arabic additive were very similar and comparable to the Total cell counts/g in formulations 27-30 of the group with Glucidex® IT 12—gum arabic additive. These results demonstrated that cell growth was not induced under the conditions tested even though the fermentable carbohydrate was supplied in excess and holding time was prolonged to 8 hours.

TABLE 19
Total cell counts in frozen formulated cell concentrates.
Results are presented as averages (n = 4)
with coefficient of variation in parentheses.

| Formulated cell concentrate | Total cells/g | |
|---|---|---|
| 23 | 1.23E+11 | (2.5%) |
| 24 | 1.25E+11 | (2.8%) |
| 25 | 1.26E+11 | (8.3%) |
| 26 | 1.22E+11 | (4.6%) |
| 27 | 1.17E+11 | (2.8%) |
| 28 | 1.35E+11 | (13.5%) |
| 29 | 1.23E+11 | (4.3%) |
| 30 | 1.22E+11 | (2.2%) |

Freeze-dried products made from frozen formulated cell concentrates 23-30 were analysed for CFU immediately after freeze-drying (Table 20). The results indicated that capability of cells to survive the freeze-drying was affected by duration of the holding time in case of glucose—gum arabic formulated cell concentrates. Slightly higher viable cell counts were measured in freeze-dried products 23-25, with the holding time from 15 min-4 hours, than in freeze-dried product 26, with 8 h holding time. The Glucidex® IT 12—gum arabic formulated freeze-dried products 27-30, had very similar CFU counts and no correlation between the potencies and holding time periods was detected for cell survival during freeze-drying.

TABLE 20
CFU counts in freeze-dried products

| Freeze-dried granulate | CFU/g |
|---|---|
| 23 | 1.8E+11 |
| 24 | 1.9E+11 |
| 25 | 1.8E+11 |
| 26 | 1.2E+11 |
| 27 | 1.2E+11 |
| 28 | 1.2E+11 |
| 29 | 1.1E+11 |
| 30 | 1.1E+11 |

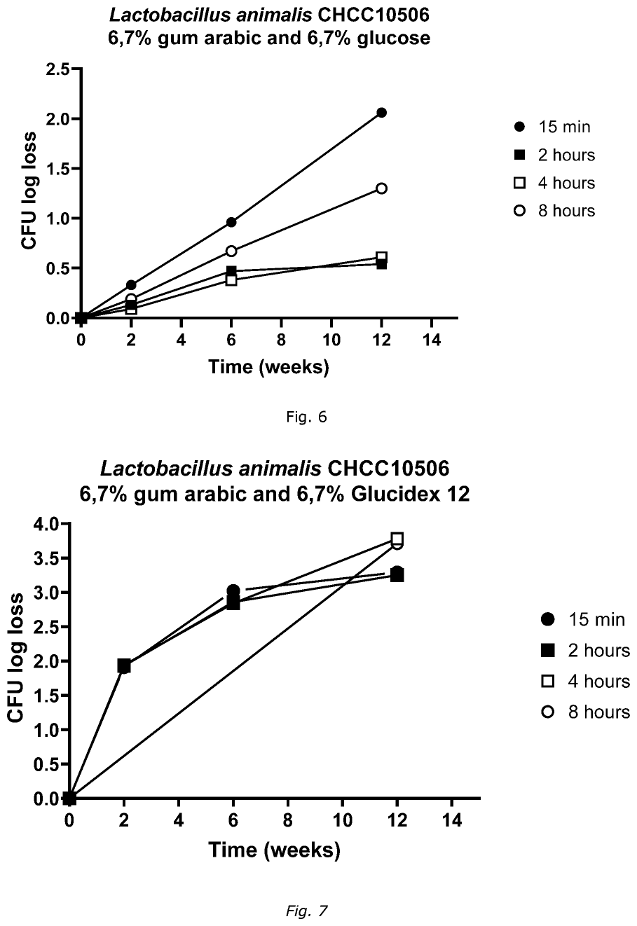

Storage stability of the eight freeze-dried products was characterized (FIGS. 6 and 7). In general, the freeze-dried products 23-26 with glucose—gum arabic additive were more stable than the freeze-dried products 27-30 with Glucidex® IT 12—gum arabic additive. After 12 weeks of the stability test at 37° C., the first group of products exhibited CFU losses of 0.5-2.1 log units, while the CFU loss in the second group of products was in range 3.3-3.8 log units.

FIG. 6 shows that, the duration of holding time made a difference for the stability of the glucose-containing freeze-dried products. The optimum holding time embraces the 2 hour and 4 hour time points, followed by 8 hour point. The freeze-dried product 23, made from formulated concentrate with 15 min holding time, had the lowest stability among the four glucose-containing freeze-dried products. This finding is linked to the fact that even though sufficient amount of fermentable sugar was present, the short term exposure of cells to the additive did not allow to energize the cells, activate metabolism and induce generation of the protective, stabilizing compounds. FIG. 7 shows that the addition of Glucidex® IT 12—gum arabic additive did not have any positive effect on storage stability. The stabilities of freeze-dried products 27-30 were poor. The 12 weeks results of the stability test revealed that prolongation of the holding time during formulation of cell concentrate to 4 h and 8 h had even more negative effect on the stability of freeze-dried products than the shorter holding time.

1. A method of preparing a frozen or dried product comprising an asporogenous prokaryote, the method comprising the steps of:
(i) combining a cell concentrate of the prokaryote with a medium comprising at least one carbohydrate, the carbohydrate being fermentable by the prokaryote, to form a pre-processing composition;
(ii) holding the pre-processing composition for a period of time and at a temperature sufficient to allow the prokaryote to ferment the fermentable carbohydrate and form an activated composition;
(iii) optionally adding a protective compound to the activated composition; and
(iv) preserving the activated composition by
(a) freezing the activated composition to form a frozen prokaryote product;
(b) drying the activated product to form a dried prokaryote product; or
(c) freezing the activated composition to form a frozen prokaryote intermediate product and then lyophilising the intermediate product to form a freeze-dried prokaryote product.

2. A method according to claim 1 wherein the carbohydrate is one or more of: a monosaccharide such as glucose, fructose, galactose or mannose; a disaccharide such as sucrose, trehalose, maltose or lactose; a sugar alcohol such as inositol; a trisaccharide such as maltotriose or raffinose; an oligosaccharide such as a fructooligosaccharide or such as a maltodextrin with DE 3-20; and a polysaccharide such as starch or inulin.

3. A method according to claim 1 or 2 wherein the total concentration of the carbohydrate in the pre-processing composition is 1-90% w/w, preferably 1-50% w/w, 1-20% w/w, 1-15% w/w, or 1-10% w/w.

4. A method according to any of claims 1 to 3 wherein the protective compound is a cryoprotectant and/or a lyoprotectant and/or a storage stabiliser, such as gum arabic, a maltodextrin, starch, pectin, cellulose, xylan, or a polyol such as glycerol, sucrose, trehalose or maltose, a protein such as gelatin, a peptide such as are supplied by yeast extract, an amino acid such as proline or a sugar alcohol such as sorbitol, mannitol or inositol.

5. A method according to any of claims 1 to 4 wherein the concentration of the protective compound in the pre-processing composition is 5-90% w/w, preferably 5-50% w/w, 5-30% w/w, 5-15% w/w, or 5-10% w/w.

6. A method according to any of claims 1 to 5 wherein the carbohydrate is glucose and the protective compound is gum arabic, and the combined concentration of the carbohydrate and the protective compound in the pre-processing composition is preferably 5-20% w/w, for example 10-20% w/w, more preferably 12-15% w/w and most preferably about 13-14% w/w.

7. A method according to any of claims 1 to 6 wherein the carbohydrate is glucose and the protective compound is gum arabic, the concentration of the carbohydrate in the pre-processing composition is 1-12%, preferably 1-10%, and the concentration of the gum arabic in the pre-processing composition is 10-35% w/w, preferably 10-22% w/w, more preferably 10-15% w/w.

8. A method according to any of claims 1 to 7 wherein step (ii) lasts for 0.5 to 8 hours, preferably 2-4 hours.

9. A method according to any of claims 1 to 8 wherein step (ii) is carried out at 4° C. to 20° C., preferably 10-15° C.

10. A method according to any of claims 1 to 9 wherein, at the end of step (ii), the hydrophobicity of the cell surface is at least 20%, preferably at least 50%, and/or has increased (compared with the hydrophobicity at the start of step (ii)) by at least 20%, preferably at least 30%, 40%, 50%, 60%, 70%, 80%, 90% or 100%, as measured by the MATH method at 22° C. and expressed as [(Initial OD₆₀₀−Final OD₆₀₀)/Initial OD₆₀₀]*100 when measured with a Φ[V_{H}/V_{B}] at at least one point between 0.01 and 1.0 and the initial OD₆₀₀ (nm) is 0.5.

11. A method according to any of claims 1 to 9 wherein, during step (ii), the pH decreases to no more than 4.5, preferably no more than 4.0, and/or decreases by at least 0.1 pH units, preferably at least 0.2, 0.5, 1.0, 1.5 pH or 2.0 units.

12. A method according to any of claims 1 to 9 wherein, during step (ii), the isoelectric point (pI) of the cells increases to at least 3.0, preferably at least 3.3, and/or increases by at least 0.1, preferably at least 0.2, 0.5, 1.0, 1.5 pH or 2.0 units, but in either case at the end of step (ii) is preferably less than 3.8, still more preferably less than 3.6 or less than 3.5.

13. A method according to any of claims 1 to 12 wherein, at the end of step (ii), a stabilising amount of the fermentable carbohydrate is still present in the activated composition.

14. A method according to any of claims 1 to 13 wherein a non-fermentable protective compound is added at the start of or during step (ii).

15. A method according to any of claims 1 to 14 wherein the frozen prokaryote product or the frozen prokaryote intermediate product has a dry weight ratio of the final additive, i.e. a sum of medium comprising at least one fermentable carbohydrate from step (i) and the protective compound from step (iii), to cell concentrate of between 6:1 and 0.3:1, preferably between 3:1 and 0.5:1 and most preferably between 2:1 and 1:1.

16. A method according to any of claims 1 to 15 wherein the prokaryote is a fermentative bacterium
from the phylum Firmicutes, such as:
a lactic acid bacterium (LAB), preferably of a genus selected from the group consisting of Streptococcus (such as Streptococcus thermophilus), Lactococcus (such as Lactococcus lactis), Oenococcus (such as Oenococcus oeni), Leuconostoc (such as species Leuconostoc mesenteroides, Leuconostoc pseudomesenteroides), Lactobacillus, Limosilactobacillus, Lacticaseibacillus, Ligilactobacillus, Lacticasei-bacillus, Lacticaseibacillus, Lactiplantibacillus, Limosilactobacillus, Ligi-lactobacillus, Lentilactobacillus, Latilactobacillus, Corripanilactobacillus, Latilactobacillus and Lactiplantibacillus; Eubacterium (such as Eubacterium limosum, Eubacterium aggregans, Eubacterium barkeri, Eubacterium lentum), Roseburia (Roseburia intestinalis, Roseburia hominis, Roseburia inulinivorans, Roseburia faecis and Roseburia cecicola), Faecalibacterium (such as species Faecalibacterium prausnitzii), Anaerostipes (such as Anaerostipes caccae), Anaerobutyricum (such as species Anaerobutyricum hallii, Anaerobutyricum soehngenii)
from the phylum Actinobacteria, such as genus Bifidobacterium (such as species Bifidobacterium animalis, Bifidobacterium longum, Bifidobacterium adolescentis, Bifidobacterium breve), genus Propionibacterium (such as species Propionibacterium freudenreichii), Cutibacterium (such as Cutibacterium acnes)
from the phylum Bacteroidetes, such as genera Bacteroides (such as species Bacteroides fragilis, Bacteroides xylanisolvens), genus Prevotella (such as species Prevotella copri) or Alistipes, or
from the phylum Verrucomicrobia, such as an Akkermansia (such as species Akkermansia muciniphila).

17. A method according to claim 16 wherein the prokaryote is one or more of: Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermentum, Ligilactobacillus animalis, Lentilactobacillus buchneri, Latilactobacillus curvatus, Companilactobacillus futsaii, Latilactobacillus sakei subsp. sakei, Lactiplantibacillus pentosus, Levilactobacillus brevis, Lactobacillus delbrueckii subsp. bulgaricus, Lactobacillus delbrueckii subsp. lactis, Lactobacillus gasseri, Lactobacillus johnsonii, Lactobacillus helveticus and Lactobacillus acidophilus, Lactobacillus jensenii, and Lactobacillus iners.

18. A frozen or dried product comprising a non-sporulating prokaryote, obtainable by a method according to any of the preceding claims.

19. A frozen or dried product according to claim 18 wherein the potency of the frozen product is 1E+09 to 1E+12 CFU/g; and the potency of the dried product is 1E+09 to 1E+13 CFU/g.
